(19) United States
(12) Patent Application Publication
(10) Pub. No.: US 2026/0193164 A1
(43) Pub. Date: Jul. 9, 2026

(54) FULLERENE DERIVATIVE COMPOSITION AND PHOTOVOLTAIC CONVERSION ELEMENT

(71) Applicant: Mitsubishi Corporation, Tokyo (JP)

(72) Inventor: Takeshi IGARASHI, Tokyo (JP)

(21) Appl. No.: 19/129,748

(22) PCT Filed: Nov. 14, 2023
(86) PCT No.: PCT/JP2023/040948

§ 371 Date: May 14, 2025

(30) Foreign Application Priority Data
Nov. 17, 2022 (JP) 2022-183843

(51) Int. Cl. C07C 69/616; H10K 30/50

(52) U.S. Cl. CPC C07C 69/616; H10K 30/50

(57) ABSTRACT

A composition of fullerene derivatives is used. The composition contains stereoisomers each containing a partial structure represented by a general formula (1) below and having a mirror image relation with each other attributable to an asymmetric carbon atom represented by C* in the general formula (1).

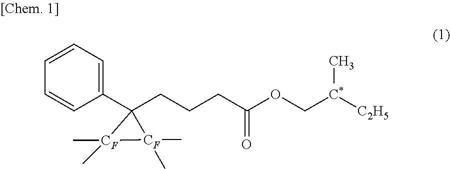

(In the formula (1), C_{F} represents carbon atoms adjacent to each other and forming a fullerene skeleton, and C* represents the asymmetric carbon atoms.)

### TECHNICAL FIELD

The present invention relates to a fullerene derivative composition and an organic photovoltaic conversion element

### BACKGROUND ART

Organic thin-film solar cells formed by providing an organic semiconductor film having electron-donating and electron-accepting properties between two different types of electrodes have the advantages that they can be produced with a simpler process and can have increased area coverage at lower costs than can inorganic solar cells represented by silicon and the like. However, it has been difficult to put organic thin-film solar cells into practical use because of their lower photovoltaic conversion efficiency, and in particular, their poorer durability than those of inorganic solar cells.

Various structures of organic thin-film solar cells have been proposed, such as Schottky type, hetero pn junction type, bulk heterojunction type, and the like. In particular, since the development of phenyl-C₆₁-butyric acid methyl ester ([60] PCBM) (NPL 1), which has an improved compatibility with electron-donor polymers, many studies on bulk heterojunction-type organic thin-film solar cells have been conducted.

For example, many [60] PCBM analogues, in which the methyl group of [60] PCBM is changed to other alkyl groups, have been synthesized. NPL 2 uses [60] PCBM analogues, in which the methyl group of [60] PCBM is changed to C₄, C₈, C₁₂, and C₁₆ linear or branched alkyl groups, in organic thin-film solar cells, and NPL 3 uses PCBM analogues, in which the methyl group of [60] PCBM is changed to C₆ and C₁₂ linear alkyl groups, in organic thin-film solar cells. It has been found that the photovoltaic conversion efficiency of organic thin-film solar cells using the same polymer is significantly affected depending on these analogues, irrespective of them having almost the same reduction potential and UV/visible light absorption characteristics.

For example, NPL 2 resulted in better organic thin-film solar cell characteristics in a case of a butyl group (C₄), and so did NPL 3 in a case of a hexyl group (C₆), compared to the case of the raw material [60] PCBM (in which the ester is a methyl group). These results seem to be related to the effect of the alkyl group difference on the interaction between fullerene derivatives and polymers, and to crystallinity variation between fullerene derivatives themselves.

PTL 1 proposes [60] PCBM analogues having an optically active center, and reports that the photovoltaic conversion efficiency and durability of organic thin-film solar cells using them are higher than those of [60] PCBM having no optically active center.

However, the durability of organic thin-film solar cells using these [60] PCBM analogues cannot be regarded to be sufficient for practical use.

### CITATION LIST

### Patent Literature

PTL 1: Japanese Patent Application Laid-Open Publication No. 2010-135665
PTL 2: Japanese Patent Application Laid-Open Publication No. 2012-201618
PTL 3: International Publication No. WO 2016/194630
PTL 4: Japanese Patent Application Laid-Open Publication No. 2008-16834

### Non-Patent Literature

NPL 1: J. Org. Chem., vol. 60, p. 532 (1995)
NPL 2: J. Phys. Chem. B, vol. 108, pp. 11921-11926 (2004)
NPL 3: T. Arai et al., IEEE 4th World Conference on Photovoltaic Energy Conversion (May 2006)

### SUMMARY OF THE INVENTION

### Technical Problem

An object of the present invention is to provide an organic photovoltaic conversion element having an improved durability, and a composition of a fullerene derivative for configuring the same.

### Solution to the Problem

The present invention provides the following solutions to the above problems.
[1] A composition of fullerene derivatives, the composition including:
   stereoisomers each containing a partial structure represented by a general formula (1) below, the stereoisomers having a mirror image relation with each other attributable to an asymmetric carbon atom represented by C* in the general formula (1),

### [Chem. 1]

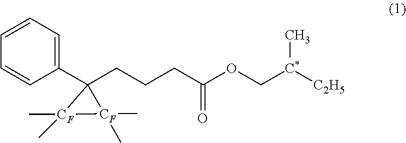

(in the formula (1), Cr represents carbon atoms adjacent to each other and forming a fullerene skeleton, and C* represents the asymmetric carbon atom).
[2] The composition according to [1],
   wherein a number of the partial structures represented by the formula (1) in one fullerene skeleton is one.
[3] The composition according to [1] or [2],
   wherein a ratio by number of moles between the stereoisomers having the mirror image relation with each other is in a range of 40:60 to 60:40.
[4] The composition according to any one of [1] to [3],
   wherein the fullerene skeleton is C₆₀, C₇₀, C₇₄, C₇₆, or C₇₈.
[5] A photovoltaic conversion element, including:
   a first electrode and a second electrode facing each other; and
   an organic layer positioned between the two electrodes,
   wherein the organic layer contains the composition of any one of [1] to [4].

### Advantageous Effects of the Invention

By using a composition of fullerene derivatives of the present invention, it is possible to obtain a photovoltaic conversion element having a high durability.

### DETAILED DESCRIPTION OF THE INVENTION

The configuration of an embodiment of the present invention will be described below. The present invention may be carried out with appropriate modifications as long as the spirit thereof is not changed.

### [Composition of Fullerene Derivative]

A composition of fullerene derivatives of the present embodiment includes stereoisomers each containing a partial structure represented by a general formula (1) below, the stereoisomers having a mirror image relation with each other attributable to an asymmetric carbon atom represented by C* in the general formula (1),

### [Chem. 2]

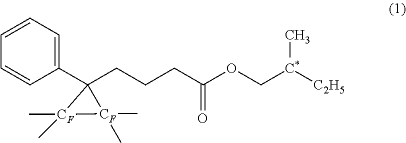

(in the formula (1), C_{F} represents carbon atoms adjacent to each other and forming a fullerene skeleton, and C* represents the asymmetric carbon atom).

In this embodiment, the term “fullerene derivative” means a compound having a structure in which a specific group is added to a fullerene skeleton, and the term “fullerene skeleton” means a carbon skeleton constituting a closed-shell structure derived from fullerene.

A fullerene derivative in the composition contains, in its side chain, a 2-methyl-1-butyl group containing an asymmetric carbon atom. Therefore, the fullerene derivative has an isomer that has a mirror image relation with itself attributable to the steric structure of the 2-methyl-1-butyl group. Here, if fullerene derivatives are only either one type of the isomers having the mirror image relation with each other, it is less difficult for the molecules to be packed, and for the fullerene derivatives to be crystallized when they are used in an organic thin-film solar cell, thereby lowering the durability of the organic thin-film solar cell. Therefore, the composition of fullerene derivatives of the present embodiment is a mixture containing stereoisomers having a mirror image relation with each other attributable to the asymmetric carbon atom on the 2-methyl-1-butyl group. Thus, it is difficult for the fullerene derivatives to be crystallized when the composition is used in an organic photovoltaic conversion element, leading to improvement of the durability of the organic photovoltaic conversion element.

The compositional ratio (ratio by number of moles) between the stereoisomers in the composition of fullerene derivatives of the present embodiment is not particularly limited, yet is preferably in the range of 40:60 to 60:40 from the viewpoint of ease of procuring the raw material, and more preferably as close to 50:50 as possible. The compositional ratio between the stereoisomers can be determined by high performance liquid chromatography (chiral HPLC) using typical chiral separation columns.

It is preferable that the number of carbon atoms in a fullerene skeleton in a fullerene derivative is 60 to 200. Specific examples include C₆₀, C₇₀, C₇₆, C₇₈, C₈₂, C₈₄, C₉₀, C₉₄, C₉₆, C₁₂₀, C₂₀₀, and the like. Among them, C₆₀, C₇₀, C₇₄, C₇₆ of C₇₈ is more preferred, C₆₀ or C₇₀ is yet more preferred, and C₆₀ is particularly preferred. This is because a raw material fullerene containing a lower number of carbon atoms is easier to procure with a higher purity, and Coo is easier to procure with a higher purity than other fullerenes.

It is preferable that the number of partial structures represented by the formula (1) in one fullerene skeleton is large, from the viewpoint of increasing solubility, whereas it is preferable that the number of partial structures is small from the viewpoint of avoiding complexity in synthesis and purification. That is, the number of partial structures is most preferably 1, as long as a solubility sufficient to produce a desired photovoltaic conversion element without any trouble can be obtained with the number of partial structures being 1. As will be shown in the Examples described later, typically, a photovoltaic conversion element can be produced even with the number of partial structures being 1.

### [Method for Producing Composition of Fullerene Derivatives]

The method for producing the fullerene derivatives is not particularly limited, and it is possible to use, for example, the method described in PTL 2, that is, a method in which phenyl-Ce-butyric acid methyl ester ([60] PCBM) is used as a raw material fullerene derivative and is transesterified with 2-methyl-1-butyl alcohol in the presence of a base catalyst.

According to this method, it is optional whether to obtain the composition of fullerene derivatives of the present embodiment by using a mixture of enantiomers of 2-methyl-1-butyl alcohol, or to obtain the composition of fullerene derivatives of the present embodiment by separately preparing fullerene derivative isomers having a mirror image relation with each other by separately using enantiomers of 2-methyl-1-butyl alcohol, and then mixing these fullerene derivatives. The former method is preferable from the viewpoint of reducing the number of steps.

### [Photovoltaic Conversion Element]

A photovoltaic conversion element of the present embodiment includes a first electrode and a second electrode facing each other, and an organic layer positioned between the two electrodes. The organic layer contains the composition of fullerene derivatives of the present embodiment. The organic layer may contain any other compound in addition to the composition of fullerene derivatives. The first electrode and the second electrode are not particularly limited, and a known material or the like can be used. The structure of the photovoltaic conversion element of the present embodiment is not particularly limited as long as the photovoltaic conversion element has the above-described features. Examples of the structure of the photovoltaic conversion element include the element structure described in PTL 3 and the like.

Although the embodiments have been described above, the above embodiments are presented as examples, and the present invention is not limited by the above embodiments. The above embodiments can be implemented in various other modes, and various combinations, omissions, substitutions, modifications, and the like are applicable without departing from the spirit of the invention. These embodiments and modifications thereof are included in the scope and spirit of the invention, as well as in the scope of the invention described in the claims and equivalents thereof.

### EXAMPLES

The embodiments will be specifically described below by way of Examples. However, the embodiments are not limited thereto.

### (Synthesis Example) Synthesis of Compound 1

Here, 2.0 g of [60] PCBM (nanom spectra E100 obtained from Frontier Carbon Corporation) as a raw material fullerene derivative was dissolved in 50 mL of orthodichlorobenzene (ODCB), and was caused to undergo a reaction at room temperature for two hours with the addition of 2.32 g (an equivalent weight of 12) of DL-2-methyl-1-butyl alcohol obtained from Tokyo Chemical Industry Co., Ltd. as a raw material alcohol, 0.05 g (an equivalent weight of 0.2) of tert-butoxypotassium as a base catalyst, and 10 g (a five-times mass) of MS-4A (molecular sieve 4A) as a porous adsorbent. The progress of the reaction was confirmed by High Performance Liquid Chromatography (HPLC). As a result, the transesterification rate of PCBM was found to be 99.3%. Solids such as the MS-4A and the like were removed by vacuum filtration using a quantitative filter paper, and the filtrate was purified through silica gel columns using ODCB as an eluent. A fraction of the purified product was concentrated to 36 mL, 150 mL of methanol was added thereto, and the precipitate was recovered by vacuum filtration using a quantitative filter paper. The filtered wet cake was dried using a vacuum dryer at 150° C. for approximately 15 hours, to obtain the following compound 1 in the form of a black solid at a purity of 99.2% and a yield of 1.51 g (a yield rate of 71%).

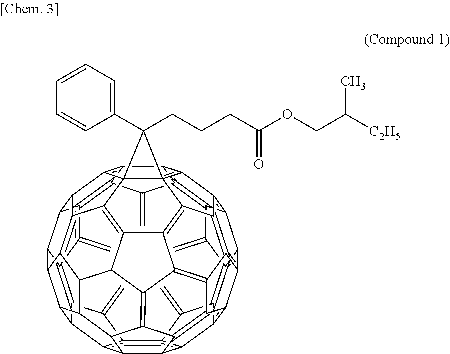

As a result of determination by chiral HPLC, it was indicated that the compound 1 was a mixture of stereoisomers having a mirror image relation with each other at a ratio of 50:50 by number of moles (racemic mixture). That is, this mixture (Compound 1) was the composition of fullerene derivatives of the present embodiment.

### (Comparative Synthesis Example) Synthesis of Compound 2

A synthesis was performed in the same manner as in Synthesis Example 1 except that(S)-(−)-2-methyl-1-butyl alcohol obtained from Tokyo Chemical Industry Co., Ltd. was used as a raw material alcohol, to obtain a compound 2 in the form of a black solid at a purity of 99.0% and a yield of 1.60 g (a yield rate of 75%). As a result of determination by chiral HPLC, it was indicated that the compound 2 was a single stereoisomer.

### Example 1

### <Preparation of Organic Photovoltaic Conversion Element 1>

An indium-tin oxide (ITO) transparent conductive film having a thickness of 110 nm deposited on a glass substrate (having a sheet resistance of 13Ω/□) was patterned to a width of 2 mm using typical photolithography and hydrochloric acid etching, to form a transparent electrode (positive electrode).

The patterned transparent electrode was cleaned by ultrasonic cleaning with a surfactant and ultrapure water, and ultrasonic cleaning with ultrapure water in this order, and then dried with a nitrogen blow. Ultimately, ultraviolet ozone cleaning was performed.

The transparent substrate was spin-coated with a conductive polymer Baytron P4083 (obtained from Starck-Vtech Ltd.) to a film thickness of 40 nm, and then heated and dried at 140° C. in the open air for 10 minutes.

After the layer of the Baytron P4083 was formed and the product was conveyed to a nitrogen atmosphere, the substrate was heated in the nitrogen atmosphere at 180° C. for 3 minutes. Next, a solution in which 1.2% by mass of a BP-1 precursor synthesized by the method described in PTL 4, and 1.0 mass % of the compound 1 (having a ratio by number of moles of 50:50 between the stereoisomers) serving as an n-type semiconductor material were dissolved was prepared as a bulk heterojunction layer, and filtrated through a 0.45 μm filter. Subsequently, the substrate was spin-coated with the solution to a thickness of 70 nm, and heated at 180° C. for 20 minutes, to obtain an i-layer. Because the molecular weight of the BP-1 precursor becomes approximately five-sixths when the BP-1 precursor changes to BP-1, the ratio of the p-type semiconductor material to the n-type semiconductor material was 1:1.

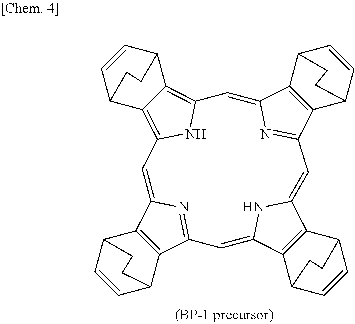 [SMILES: C1=CC2CCC1C1=C2c2cc3[nH]c(cc4nc(cc5[nH]c(cc1n2)c1c5C2C=CC1CC2)C1=C4C2C=CC1CC2)c1c3C2C=CC1CC2]

The substrate prepared up until having the bulk heterojunction layer was moved to a vapor deposition apparatus without being exposed to the open air, and the element was set such that a shadow mask having a width of 2 mm would be orthogonal to the transparent electrode. Then, the pressure was reduced to 4×10⁻⁴ Pa. Bathocuproine obtained from Sigma-Aldrich Co. LLC and aluminum were put in a tantalum resistor thermal boat and a tungsten resistor thermal boat, respectively, and the boats were mounted on the vapor deposition apparatus.

Next, the tantalum resistor thermal boat was turned on and heated, to form an electron transport layer of bathocuproine (BCP) to have a thickness of 6 nm on the substrate. Next, the tungsten tantalum thermal boat was turned on and heated, to vapor-deposit a negative electrode having a film thickness of 100 nm at a vapor deposition rate of 1 nm/sec to 2 nm/sec such that the negative electrode would be orthogonal to the transparent conductive film, to obtain an organic photovoltaic conversion element 1 in the form of a square having a size of 2 mm square.

After being sealed with an aluminum cap and a UV-curable resin (UV RESIN XNR5570-B1 obtained from Nagase ChemteX Corporation) in a nitrogen atmosphere, the obtained organic photovoltaic conversion element 1 was taken out to the open air and irradiated with solar simulator light having an irradiation intensity of 100 mW/cm² (AM1.5G), to measure the voltage-current characteristic and measure the initial conversion efficiency. Furthermore, the initial conversion efficiency here was regarded as 100, and the conversion efficiency after keeping irradiation at the irradiation intensity of 100 mW/cm² for 100 hours with a resistor connected between the positive electrode and the negative electrode was evaluated, to calculate a relative efficiency deterioration. The results are shown in Table 1.

### Comparative Examples 1 to 5

### <Preparation of Organic Photovoltaic Conversion Elements 2 to 6>

Organic photovoltaic conversion elements 2 to 6 were obtained in the same manner as that of the organic photovoltaic conversion element 1, except that unlike in the preparation of the organic photovoltaic conversion element 1, the n-type semiconductor material was changed to the compounds shown in Table 1. As [60] PCBM of Comparative Example 2, nanom spectra E100 obtained from Frontier Carbon Corporation was used. Compounds 3 to 5, shown below, used in Comparative Examples 3 to 5 were synthesized according to the method described in PTL 2. As the raw material alcohols, those that were suitable for the following chemical formulae of the compounds 3 to 5 were used. The raw material alcohols used in the synthesis of the compounds 3 and 4 were both linear.

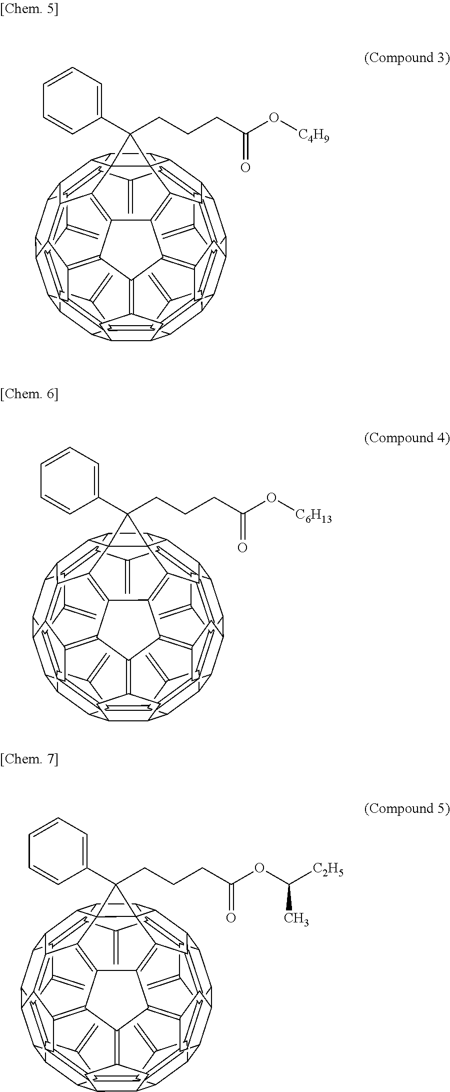

After being sealed with an aluminum can and a UV-curable resin in a nitrogen atmosphere, the obtained organic photovoltaic conversion elements 2 to 5 were taken out to the open air and irradiated with solar simulator light having an irradiation intensity of 100 mW/cm² (AM1.5G), to measure the voltage-current characteristic and measure the initial conversion efficiency. Furthermore, the initial conversion efficiency here was regarded as being 100%, and the conversion efficiency after keeping irradiation at the irradiation intensity of 100 mW/cm² for 100 hours with a resistor connected between the transparent electrode and the counter electrode was evaluated. The results are shown in Table 1.

TABLE 1

| | | | Composi- | | |
|---|---|---|---|---|---|
| | Organic | | tional | | Relative |
| | PV | n-type | ratio | Initial | eff. |
| | conversion | semicond. | between | conversion | deterio- |
| | element | material | stereoisomers | efficiency | ration |
| Ex. 1 | 1 | Compound 1 | 50:50 | 1.8% | 15% |
| Comp. | 2 | Compound 2 | 100:0 | 1.8% | 30% |
| Ex. 1 | | | | | |
| Comp. | 3 | [60] PCBM | — | 1.9% | 80% |
| Ex. 2 | | | | | |
| Comp. | 4 | Compound 3 | — | 1.9% | 45% |
| Ex. 3 | | | | | |
| Comp. | 5 | Compound 4 | — | 1.7% | 50% |
| Ex. 5 | | | | | |
| Comp. | 6 | Compound 5 | 100:0 | 1.9% | 30% |
| Ex. 6 | | | | | |

As can be seen from Table 1, it can be appreciated that the organic photovoltaic conversion element of the present embodiment has a high durability without the initial conversion efficiency being impaired.

### INDUSTRIAL AVAILABILITY

The present invention can be suitably applied to organic photovoltaic conversion elements.

This application claims priority based on Japanese Patent Application No. 2022-183843 filed with the Japan Patent Office on Nov. 17, 2022, and the entire contents of the application are incorporated herein.

1. A composition of fullerene derivatives, the composition comprising:
stereoisomers each containing a partial structure represented by a general formula (1) below, the stereoisomers having a mirror image relation with each other attributable to an asymmetric carbon atom represented by C* in the general formula (1),
[Chem. 1] 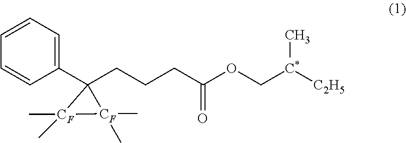
(in the general formula (1), C_{F} represents carbon atoms adjacent to each other and forming a fullerene skeleton, and C* represents the asymmetric carbon atom).

2. The composition according to claim 1,
wherein a number of the partial structures represented by the general formula (1) in one fullerene skeleton is one.

3. The composition according to claim 1,
wherein a ratio by number of moles between the stereoisomers having the mirror image relation with each other is in a range of 40:60 to 60:40.

4. The composition according to claim 1,
wherein the fullerene skeleton is C₆₀, C₇₀, C₇₄, C₇₆, or C₇₈.

5. A photovoltaic conversion element, comprising:
a first electrode and a second electrode facing each other; and
an organic layer positioned between the first electrode and the second electrode,
wherein the organic layer contains the composition of claim 1.
